(19) United States
(12) Patent Application Publication
(10) Pub. No.: US 2012/0265175 A1
(43) Pub. Date: Oct. 18, 2012

(54) MALLEABLE INSTRUMENT FOR LAPAROSCOPIC PROCEDURES

(75) Inventors: Nicolas Deflandre, Souvigny (FR); François Virmoux, Gennetines (FR)

(73) Assignee: MEDTRONIC XOMED, INC., Jacksonville, FL (US)

(21) Appl. No.: 13/087,007

(22) Filed: Apr. 14, 2011

(51) Int. Cl. A61B 17/00

(52) U.S. Cl. 606/1

(57) ABSTRACT

A laparoscopoic tool includes a handle assembly, a tool implement, an outer tube defining an inner lumen and a wire assembly positioned within the lumen of the outer tube and coupled to the handle assembly and the tool implement. The outer tube and the wire assembly are configured to be shaped to form at least one bend region.

### BACKGROUND

Laparoscopic surgery includes forming small incisions in a patient and performing a procedure through the incision using one or more laparoscopic surgical tools. Current laparoscopical surgical techniques utilize one or more trocars that establish a port for introduction of the surgical tools. One type of surgical technique is known as single incision laparoscopic surgery (“SILS”), where only a single incision and single trocar are used during the surgical procedure. In one example, SILS procedures utilize an umbilical trocar introduced through an incision in the umbilical region of a patient. As incisions in the umbilical region tend to be relatively invisible after surgery and allow for quicker recovery of the patient, SILS procedures can be a preferred method of surgery in many instances. Even with inherent benefits to utilizing the umbilical region for SILS procedures, procedures using entry through the umbilical region can be difficult due to lack of space and proximity with a targeted tissue site. As such, specialty curved or articulated tools have given physicians opportunity to have more clearance in reaching and operating upon a targeted tissue site. These curved tools can still be difficult to work with, since patients can be varied in size and varied target surgical sites can be difficult to access.

### SUMMARY

A laparoscopic surgical tool is disclosed that includes a handle assembly, a tool implement, an outer tube and an inner wire assembly coupling the handle assembly to the tool implement. The outer tube and inner wire assembly can be manipulated to conform to a desired shape.

In one embodiment, the wire assembly is positioned within an inner lumen of the outer tube. A surgical environment can be evaluated to identify a path from an incision to a targeted tissue site. Upon evaluation, the outer tube and wire assembly can be shaped to form one or more bend regions. As such, a surgeon is afforded easier access to the targeted tissue site. Subsequently, the laparoscopic tool can be withdrawn and the handle assembly and/or tool implement can be replaced with an alternative handle assembly and/or tool implement while maintaining the one or mor bend regions.

### BRIEF DESCRIPTION OF THE DRAWINGS

FIG. 1 is a side view of a laparoscopic tool in a disassembled state.

FIG. 2 is a side view of a laparoscopic tool after final assembly.

FIG. 3 is a side view of a laparoscopic tool in a first curved shape.

FIG. 4 is a side view of a laparoscopic tool in a second curved shape.

FIG. 5 is a schematic view of a surgical environment.

### DETAILED DESCRIPTION

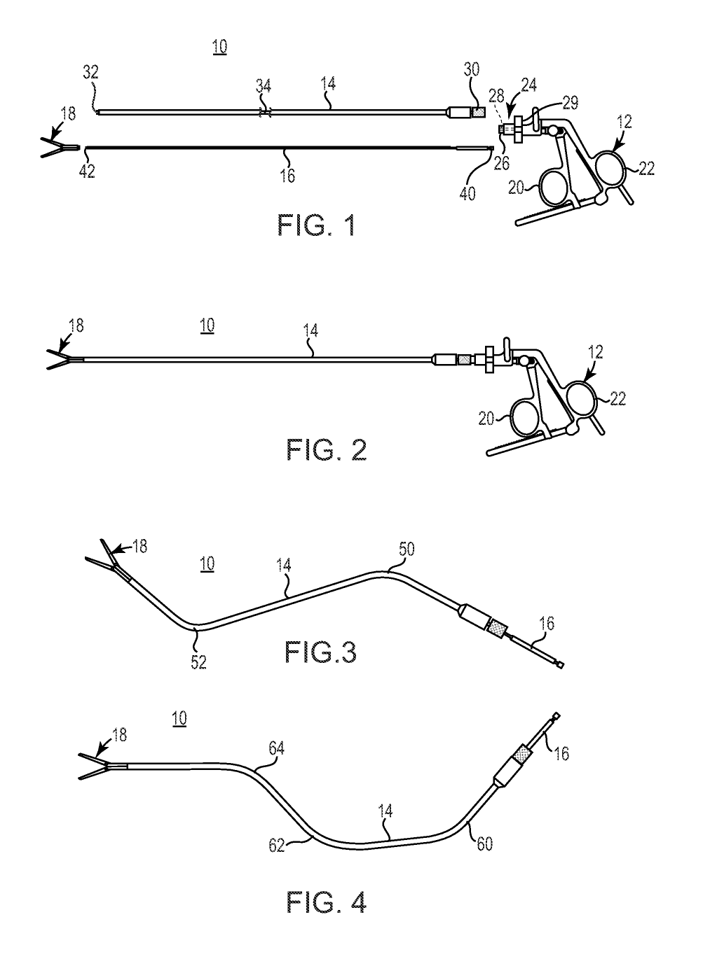

FIG. 1 is an exploded side view of a laparoscopic tool 10 whereas FIG. 2 is a side view of tool 10 in an assembled state. The tool 10 includes a handle assembly 12, an outer tube or sleeve 14, an inner wire assembly 16 and a tool implement 18. Handle assembly 12 is operably coupled to tool implement 18 through wire assembly 16. Tube 14 is coaxially arranged around wire assembly 16 and coupled to handle assembly 12. A surgeon utilizing tool 10 is able to bend tube 14 and wire assembly 16 to a desired shape to form a configuration suitable to perform a procedure. As such, the surgeon conforms the outer tube 14/wire assembly 16 to a shape that leads from an incision to a target tissue site. Tube 14 and wire assembly 16 are configured to be coupled with alternative handle assemblies and tool implements as desired. As such, the shape of the tube 14 and wire assembly 16 can be maintained to perform various tasks within a surgical environment.

Handle assembly 12 includes a trigger handle 20 and a fixed handle 22 for operation of the tool implement 18. Additionally, handle assembly 12 includes a coupling portion 24 for attachment to the tube 14 and wire assembly 16. In particular, coupling portion 24 includes a threaded shaft 26 for coupling to tube 14 and an aperture 28 for receiving wire assembly 16, which is directly coupled to trigger handle 20. In addition, handle assembly 12 includes a control knob 29, which can be utilized to effect rotational movement of wire assembly 16 and thus tool implement 18. Other handle assemblies can also be used having one or more features such as rotatable, fixed, axial, angled, with a monopolar connection, with a bipolar connection and/or combination thereof.

Outer tube 14 includes a proximal end 30 and a distal end 32 opposite the proximal end 30. Proximal end 30, in one embodiment, defines a threaded bore configured to mate with threaded shaft 26 of handle assembly 12, so as to secure tube 14 to handle assembly 12. Outer tube 14 further defines a lumen 34 (referenced generally) configured to receive the wire assembly 16. In one embodiment, tube 14 is formed of annealed 300 series stainless steel, wherein an outer diameter of the tube 14 is approximately 4.0 millimeters and a diameter of lumen 34 is approximately 2.6 millimeters. In particular, the tube 14 is formed to be malleable to a degree such that outer tube 14 can be bent to a desired shape, yet maintain the desired shape during operation of the tool 10. Other diameters/dimensions of the tube 14 can be used in alternative embodiments. In one embodiment, tube 14 can be covered with an insulating material. As desired, tube 14 can be formed in various lengths, for example to accommodate normal patients, obese patients and/or pediatric patients.

Wire assembly 16 includes a proximal end 40 and a distal end 42 positioned opposite the proximal end 40. In one embodiment, wire assembly 16 is formed of annealed 300 series stainless steel comprising a single wire having an outer diameter of approximately 1.4 to 1.8 millimeters. In an alternative embodiment, wire assembly 16 is a cable having a plurality of wires with an outer diameter of approximately 1.4 to 1.8 millimeters. In any event, wire assembly 16 is formed so as to conform to the shape of outer tube 14 and still move within lumen 34 relative to the outer tube 14 in a longitudinal direction upon actuation of handle assembly 12. To this end, wire assembly 16 can be coated with a non-sticking material, in one embodiment, so as to facilitate movement of the wire assembly 16 within lumen 34. In the embodiment illustrated, proximal end 40 defines a feature (e.g., a notch or ball) configured to be inserted into aperture 28 and coupled to trigger handle 20. Additionally, distal end 42 defines a feature (e.g., a notch or ball) configured to be coupled to tool implement 18. Wire assembly 16 is coupled to trigger handle 22 in such a way that by squeezing handle assembly 12 (i.e., by causing movement of trigger handle 20 relative to fixed handle 22), distal end 42 is actuated toward handle assembly 12. This actuation, in turn, causes operation of tool implement 18. As such, a surgeon can operate tool implement 18 remotely via actuation of the handle assembly 12. Moreover, as discussed above, control knob 29 can be rotated to cause rotation of wire assembly 16 and tool implement 18.

As discussed above, outer tube 14 and inner wire assembly 16 are formed to be malleable such that a surgeon can deform and manipulate the shape of the outer tube 14 and inner wire assembly 16. As a result, prior to inserting tool 10 into a laparoscopic port, a surgeon can evaluate a surgical area and in particular a desired approach angle from a port opening (e.g., the umbilical region) to a target surgical site. The surgeon can then form the outer tube 14 and inner wire assembly 16 to a desired shape by creating one or more bend regions therein.

Tool implement 18 is operably coupled to the handle assembly 12 through the wire assembly. As such, operation of the handle assembly 12 causes operation of the tool assembly 18. In various embodiments, tool implement 18 can take various forms in various shapes and sizes. Moreover, the tool implements can be configured to perform various functions such as grasping, cutting, clamping and/or coagulation.

As illustrated in FIG. 3, the surgeon can form tool 10 such that outer tube 14 forms a first bend region 50 and a second bend region 52 along a length of the outer tube 14. In an alternative configuration, illustrated in FIG. 4, tool 10 forms a first bend region 60, a second bend region 62 and a third bend region 64 along a length of outer tube 14. Other bend regions can be formed within tool 10 as desired. Once a desired shape is selected, tool implement 18 can be delivered to a targeted tissue site to perform a desired procedure.

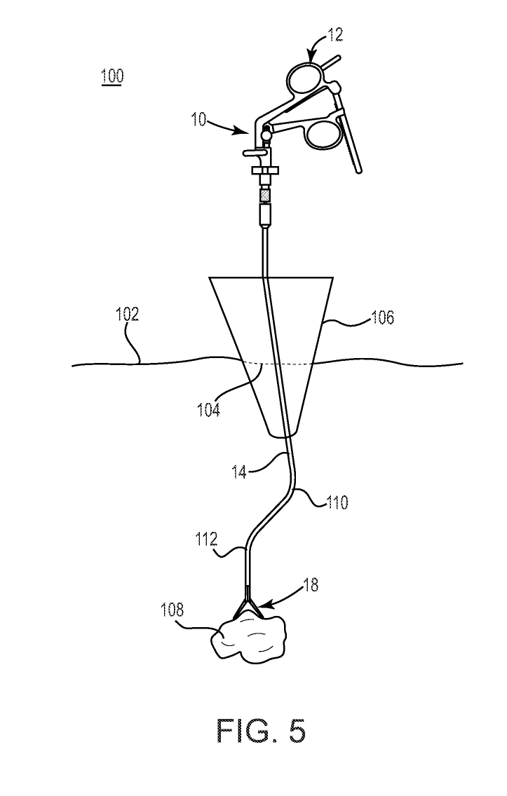

FIG. 5 is a schematic illustration of a surgical environment 100. A patient body 102 includes an incision 104 formed therein for insertion of a port 106. A surgeon desires to operate on a target tissue site 108 located within patient body 102 and remote from the incision 104. After inspection and evaluating the location of target site 108, a surgeon forms a shape of tool 10 to include one or more bend regions. In the embodiment illustrated, tool 10 is formed to include a first bend region 110 and a second bend region 112. Once the desired bend regions are formed, tool 10 is inserted into port 106 until tool implement 18 engages tissue site 108. Handle assembly 12 can then be actuated so as to operate tool implement 18. After operating tool implement 18, tool 10 can be withdrawn from the patient body 102. After withdrawal of tool 10, handle assembly 12 and/or tool implement 18 can be replaced with different handle assemblies or tool implements as desired while maintaining the shape of outer tube 14, in particular including bend region 110 and bend region 112.

Although the present disclosure has been described with reference to preferred embodiments, workers skilled in the art will recognize that changes can be made in form and detail without departing from the spirit and scope of the present disclosure.

What is claimed is:

1. A laparoscopic tool, comprising:
a handle assembly;
a tool implement;
an outer tube defining an inner lumen; and
a wire assembly positioned within the lumen of the outer tube and coupled to the handle assembly and the tool implement,
wherein the outer tube and the wire assembly are configured to be shaped to form at least one bend region.

2. The laparoscopic tool of claim 1, wherein the handle assembly includes a trigger handle and a fixed handle, wherein movement of the trigger handle relative to the fixed handle causes relative movement between the outer tube and the wire assembly.

3. The laparoscopic tool of claim 2, wherein movement of the wire assembly relative to the outer tube causes operation of the tool implement.

4. The laparoscopic tool of claim 1, wherein the handle assembly further includes a control knob, wherein rotation of the control knob causes rotation of the tool implement.

5. The laparoscopic tool of claim 1, wherein the outer tube and the wire assembly are configured to maintain the at least one bend region while the handle assembly is removed therefrom and a second handle assembly is coupled thereto.

6. The laparoscopic tool of claim 1, wherein the outer tube and the wire assembly are configured to maintain the at least one bend region while the tool implement is removed therefrom and a second tool implement is coupled thereto.

7. The laparoscopic tool of claim 1, wherein the outer tube and the wire assembly are configured to form at least two bend regions.

8. The laparoscopic tool of claim 1, wherein the outer tube and the wire assembly are configured to form at least three bend regions.

9. The laparoscopic tool of claim 1, wherein the outer tube and the wire assembly are formed of stainless steel.

10. A method of using a laparoscopic tool, comprising:
providing a tool having a handle assembly, a tool implement, an outer tube and a wire assembly coupled to the handle assembly and the tool implement; and
shaping the outer tube and wire assembly to form at least one bend region.

11. The method of claim 10, further comprising:
providing relative movement between a trigger handle and a fixed handle of the handle assembly so as to cause relative movement between the outer tube and the wire assembly.

12. The method of claim 11, wherein movement of the wire assembly relative to the outer tube causes operation of the tool implement.

13. The method of claim 10, further comprising:
providing a control knob on the handle assembly, wherein rotation of the control knob causes rotation of the tool implement.

14. The method of claim 10, further comprising:
removing the handle assembly from the wire assembly; and
attaching a second handle assembly to the wire assembly while maintaining the at least one bend region and the outer tube and the wire assembly.

15. The method of claim 10, further comprising:
removing the tool implement from the wire assembly; and
coupling a second tool implement to the wire assembly while maintaining the at least one bend region.

16. The method of claim 10, wherein shaping the outer tube and wire assembly includes forming at least two bend regions.

17. The method of claim 10, wherein shaping the outer tube and the wire assembly includes forming at least three bend regions.

18. The method of claim 10, wherein the outer tube and the wire assembly are formed of stainless steel.

19. The method of claim 10, further comprising:
inspecting a surgical environment to determine a path from an incision to targeted tissue; and
shaping the outer tube and the wire assembly to conform to the desired path.

20. The method of claim 10, wherein shaping the outer tube and wire assembly is performed by a hand of a user forming the at least one bend region.
